(19) United States
(12) Patent Application Publication
(10) Pub. No.: US 2015/0202463 A1
(43) Pub. Date: Jul. 23, 2015

(54) APPARATUS AND METHOD FOR LASER ALIGNMENT IN RADIATION THERAPY

(71) Applicant: National Cancer Center, Gyeonggi-do (KR)

(72) Inventors: Young Kyung Lim, Gyeonggi-do (KR); Jung Won Kwak, Gyeonggi-do (KR); Yeon-Joo Kim, Gyeonggi-do (KR); Sung Ho Moon, Seoul (KR); Dong Ho Shin, Gyeonggi-do (KR); Se Byeong Lee, Gyeonggi-do (KR); Kwanghyun Jo, Gyeonggi-do (KR); Ui-Jung Hwang, Gyeonggi-do (KR)

(21) Appl. No.: 14/483,466

(22) Filed: Sep. 11, 2014

(30) Foreign Application Priority Data
Jan. 23, 2014 (KR) 10-2014-0008393

(51) Int. Cl. A61N 5/10

(52) U.S. Cl. CPC A61N 5/1049; A61N 5/1081; A61N 2005/105

(57) ABSTRACT

In an apparatus for laser alignment and a method of aligning laser using the same, the apparatus for laser alignment is used in a radiation therapy system. The radiation therapy system includes a rotatable gantry having a radiation source generating radiation, a radiation shaping unit and a crosshair indicating a radiation field center, a couch on which a patient is disposed and a laser device disposed adjacent to the gantry to irradiate a laser for aiming. A light source irradiates light being mounted in the gantry. The couch is disposed adjacent to the gantry. Position and direction of the couch are controllable. The apparatus for laser alignment includes the plural alignment marks spaced apart from each other to be projected, thereby simultaneously checking alignment status by the irradiation of the light in at least one direction; and a level and a level adjusting part.

### PRIORITY STATEMENT

This application claims priority under 35 U.S.C. §119 to Korean Patent Application No. 10-2014-0008393, filed on Jan. 23, 2014 in the Korean Intellectual Property Office (KIPO), the contents of which application are herein incorporated by reference in their entirety.

### BACKGROUND

1. Field of Disclosure

The present disclosure of invention relates to an apparatus and method for laser alignment in radiation therapy.

2. Description of Related Technology

An apparatus for radiation treatment and the laser apparatuses for aiming treatment site are provided in a care unit for cancer treatment.

An apparatus for radiation treatment, in general, is C-shaped gantry, and the laser apparatuses are mounted on the walls of the care unit. The position and direction of the laser apparatus can be changed for alignment.

The treatment site of a patient, onto which the radiation will be irradiated, is checked by a radiation therapy simulation system to be marked on a patient's skin by an ink, a tattoo, etc. In the radiation care unit, the laser is aimed onto the treatment site that is marked by the ink, the tattoo, etc., so that the radiation is precisely irradiated onto the treatment site.

The laser apparatus for aiming the treatment site requires high accuracy for accurate treatment. Aligned position or direction may be changed by internally or externally provided vibration or impact of the care unit, vibration of a building, automobile transportation, earthquake, etc. In order to solve the above-mentioned problem, the alignment of the laser is repetitively checked and calibrated. An apparatus for laser alignment is used to checking and calibrating the laser.

However, a conventional apparatus or method for laser alignment uses only a single reference to align the laser, so that alignment error is big and time for checking and calibrating the laser is long. Also, an isocenter of the radiation, which is very important in the radiation treatment, is not used as a reference of the laser alignment.

Related arts are Korean patent laid open publication number 2006-0135063 (entitled “Patient Positioning Assembly” on Dec. 28, 2006) and U.S. Pat. No. 7,559,693 (entitled “Method and Apparatus for X-ray Alignment” on Jul. 14, 2009).

### SUMMARY

The present invention is developed to solve the above-mentioned problems of the related arts. The present invention provides an apparatus and method for laser alignment in radiation therapy, which are capable of aligning laser at high accuracy and greatly decreasing time for aligning.

According to the exemplary embodiment of the present invention, an apparatus for alignment used in a radiation therapy system is provided. The radiation therapy system includes a rotatable gantry having a radiation source generating radiation, a radiation shaping unit and a crosshair indicating a radiation field center, a couch on which a patient is disposed and a laser device disposed adjacent to the gantry to irradiate a laser for aiming. A light source irradiates light being mounted in the gantry. The couch is disposed adjacent to the gantry. Position and direction of the couch are controllable. The apparatus for alignment includes the plural alignment marks spaced apart from each other to be projected, thereby checking alignment status by the irradiation of the light in at least one direction; a level indicating horizontal level of the alignment marks; and a level adjusting part adjusting horizontal level of the alignment marks.

In an example embodiment, the apparatus for alignment may further include a radiation imaging part including the plural radiation imaging planes to check an isocenter of the radiation irradiated by the gantry.

In an example embodiment, the radiation imaging plane may include a first imaging plane directly facing a rotation plane of the gantry or facing the rotation plane of the gantry by rotation; and a second imaging plane directly facing a rotation plane of the radiation shaping unit or facing the rotation plane of the radiation shaping unit by rotation.

In an example embodiment, the radiation imaging part may include or receive at least one of radiation image detectors, such as a gel dosimeter, a fluorescent substance, a scintillator and an imaging device using a photovoltaic effect. The radiation may be irradiated onto the radiation imaging part through the radiation shaping unit.

In an example embodiment, the laser device may include first and second laser units disposed on left and right of the gantry. The apparatus for alignment may further include first and second plates facing each other. The alignment mark may include a first alignment mark formed on the first plate and a second alignment mark formed on the second plate. The first and second alignment marks may be used for aligning the first and second laser units with the light source mounted in the gantry.

In an example embodiment, the alignment mark may have a crosshair shape, and the first and second alignment marks may have different linear shapes different from each other.

In an example embodiment, the apparatus for alignment may further include an alignment light source irradiating light toward the alignment mark.

In an example embodiment, the laser device may further include a third laser unit disposed on a side facing the gantry. The apparatus for alignment may further include a third plate nonparallel from the first and second plates and facing the third laser. The alignment mark may further include a third alignment mark formed on the third plate. The third alignment mark may be used for aligning the third laser unit with the alignment light source.

In an example embodiment, the laser device may further include a fourth laser unit disposed above the gantry. The apparatus for alignment may further include a fourth plate nonparallel from the first to third plates and facing the fourth laser. The alignment mark may further include a fourth alignment mark formed on the fourth plate. The fourth alignment mark may be used for aligning the fourth laser unit with the alignment light source.

According to the exemplary embodiment of the present invention, a method for laser alignment used in a radiation therapy system is provided. The radiation therapy system includes a rotatable gantry having a radiation source generating radiation, a radiation shaping unit and a crosshair indicating a radiation field center, a couch on which a patient is disposed and a laser device disposed adjacent to the gantry to irradiate a laser for aiming. A light source irradiates light being mounted in the gantry. The couch is disposed adjacent to the gantry. Position and direction of the couch are controllable. The method for laser alignment includes preparing an apparatus for alignment including first and second alignment marks spaced apart from each other to be projected to simultaneously checking alignment status by the irradiation of the light in at least one direction, a level and a level adjusting part; disposing the apparatus for alignment on the couch; transporting the apparatus for alignment toward a radiation center to level the apparatus for alignment; rotating or translating the gantry, the radiation shaping unit and the couch while irradiating the light of the light source in the gantry to coincide the first and second alignment marks with an optical image of the crosshair indicating a radiation field center; and locating the laser device on an optical image formed by coinciding the first and second alignment marks, and controlling direction of the laser so that the first and second alignment marks are coincided with the laser.

In an example embodiment, the alignment mark may have a crosshair shape, and the first and second alignment marks may have different linear shapes different from each other.

In an example embodiment, the method may further include checking an isocenter of the radiation irradiated from the gantry to translate the laser or the laser device.

In an example embodiment, the apparatus for alignment may further include a radiation imaging part having the plural image surfaces, and the isocenter of the radiation irradiated from the gantry may be checked using radiation image obtained by the radiation imaging part.

In an example embodiment, the radiation imaging plane may include a first imaging plane directly facing a rotation plane of the gantry or facing the rotation plane of the gantry by rotation; and a second imaging plane directly facing a rotation plane of the radiation shaping unit or facing the rotation plane of the radiation shaping unit by rotation.

In an example embodiment, the isocenter of the radiation may be checked by rotating the gantry to irradiate the radiation onto the first imaging plane while the radiation shaping unit is fixed; and rotating the radiation shaping unit onto the second imaging plane while the gantry is fixed.

According to the exemplary embodiment of the present invention, a method for laser alignment used in a radiation therapy system is provided. The radiation therapy system includes a rotatable gantry having a radiation source generating radiation, a radiation shaping unit and a crosshair indicating a radiation field center, a couch on which a patient is disposed and a laser device disposed adjacent to the gantry to irradiate a laser for aiming. A light source irradiates light being mounted in the gantry. The couch is disposed adjacent to the gantry. Position and direction of the couch are controllable. The method for laser alignment includes preparing an apparatus for alignment including first and second alignment marks spaced apart from each other to be projected to simultaneously checking alignment status by the irradiation of the light in at least one direction, a level and a level adjusting part; optically aligning the laser device using optical image obtained by lights having substantially simultaneously passed through the crosshair indicating a radiation field center, the first alignment mark and the second alignment mark; and determining center of the radiation irradiated from the gantry to align the laser device.

In an example embodiment, the alignment mark may have a crosshair shape, and the first and second alignment marks may have different linear shapes different from each other.

In an example embodiment, the apparatus for alignment may further include a radiation imaging part having the plural image surfaces, and the isocenter of the radiation irradiated from the gantry may be checked using radiation image obtained by the radiation imaging part.

In an example embodiment, the radiation imaging plane may include a first imaging plane directly facing a rotation plane of the gantry or facing the rotation plane of the gantry by rotation; and a second imaging plane directly facing a rotation plane of the radiation shaping unit or facing the rotation plane of the radiation shaping unit by rotation.

In an example embodiment, the isocenter of the radiation may be checked by rotating the gantry to irradiate the radiation onto the first imaging plane while the radiation shaping unit is fixed; and rotating the radiation shaping unit onto the second imaging plane while the gantry is fixed.

According to the present invention, the apparatus and method for laser alignment have high aligning accuracy and greatly decreased tome for aligning.

### BRIEF DESCRIPTION OF THE DRAWINGS

The above and other features and advantages will become more apparent by describing exemplary embodiments thereof with reference to the accompanying drawings, in which:

FIG. 1 is a perspective view illustrating a system for radiation therapy according to an exemplary embodiment of the present invention;

FIG. 2 is a perspective view illustrating a method for controlling size of radiation irradiation of a system for radiation therapy according to an exemplary embodiment of the present invention;

FIG. 3 is a perspective view illustrating an apparatus for alignment according to an exemplary embodiment of the present invention;

FIG. 4 is a perspective view illustrating an apparatus for alignment disposed in a system for radiation therapy according to an exemplary embodiment of the present invention;

FIG. 5 is a flow chart illustrating aligning a laser device according to an optical method of an exemplary embodiment of the present invention;

FIG. 6 is a perspective view illustrating coincidence of alignment marks of optical alignment according to an exemplary embodiment of the present invention;

FIG. 7 is a perspective view illustrating control of position and direction of a laser device according to an optical alignment of an exemplary embodiment of the present invention;

FIG. 8 is a flow chart illustrating radiation alignment according to an exemplary embodiment of the present invention;

FIG. 9 is a radiation image irradiated on a radiation film according to an exemplary embodiment of the present invention;

FIG. 10 is a side view illustrating a vertical laser alignment of a radiation alignment according to an exemplary embodiment of the present invention;

FIG. 11 is a plan view illustrating a horizontal laser alignment of a radiation alignment according to an exemplary embodiment of the present invention;

FIG. 12 is a perspective view illustrating difference of a horizontal measurement error between a related art and the present invention;

FIG. 13 is a perspective view illustrating difference of an optical error between a related art and the present invention; and

FIG. 14 is a perspective view illustrating a radiation imaging part according to an exemplary embodiment of the present invention.

### DETAILED DESCRIPTION

Hereinafter, exemplary embodiment of the invention will be explained in detail with reference to the accompanying drawings.

The following figures are examplarily shown to explain the inventive concept of the present invention more concretely, and thus the scope of the present invention is not limited by the following figures.

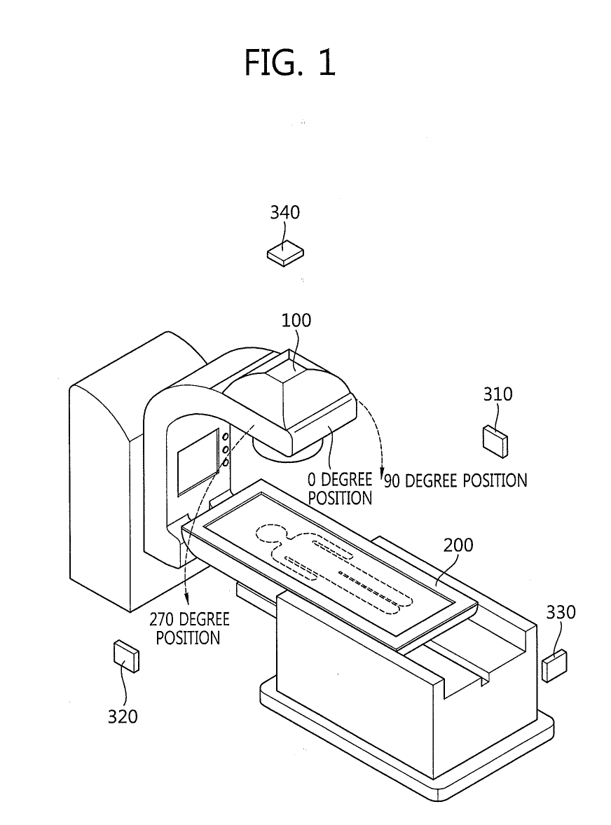

FIG. 1 is a perspective view illustrating a system for radiation therapy according to an exemplary embodiment of the present invention.

The system 1 for radiation therapy includes a gantry 100, a couch 200 and a laser device 310 to 340. The gantry 100 irradiates radiation. A patient is disposed in the couch 200. A laser is generated by the laser device 310 to 340.

The gantry 100 has a C-shape, and rotates with respect to the couch 200. The gantry 100 may rotate at a 0-degree position that is in front of the patient, a 90-degree position that is left of the patient and a 270-degree position the is right of the patient.

The patient for treatment is disposed on the couch 200. The couch 200 may transport in a horizontal direction, a vertical direction and a rotational direction.

The laser device 310 to 340 includes a first laser unit 310, a second laser unit, a third laser unit 330 and a fourth laser unit 340. When the laser device 310 to 340 is disposed on the patient as shown in FIG. 1, the first laser unit 310 is disposed on the left of the patient, the second laser unit 320 is disposed on the right of the patient, the third laser unit 330 is disposed on feet of the patient, and the fourth laser unit 340 is disposed on a ceiling.

The laser device 310 to 340 aims treatment site of the patient, and the radiation is irradiated on the treatment site to cure the patient.

Side laser units 310 and 320 irradiate vertical and horizontal lasers, and other laser units 330 and 340 irradiate sagittal laser. Each laser unit 310 to 340 is mounted and fixed on a sidewall or a ceiling, and includes a laser generating part and a driving part for transporting the laser generating part, thereby controlling position and direction of irradiation of the laser.

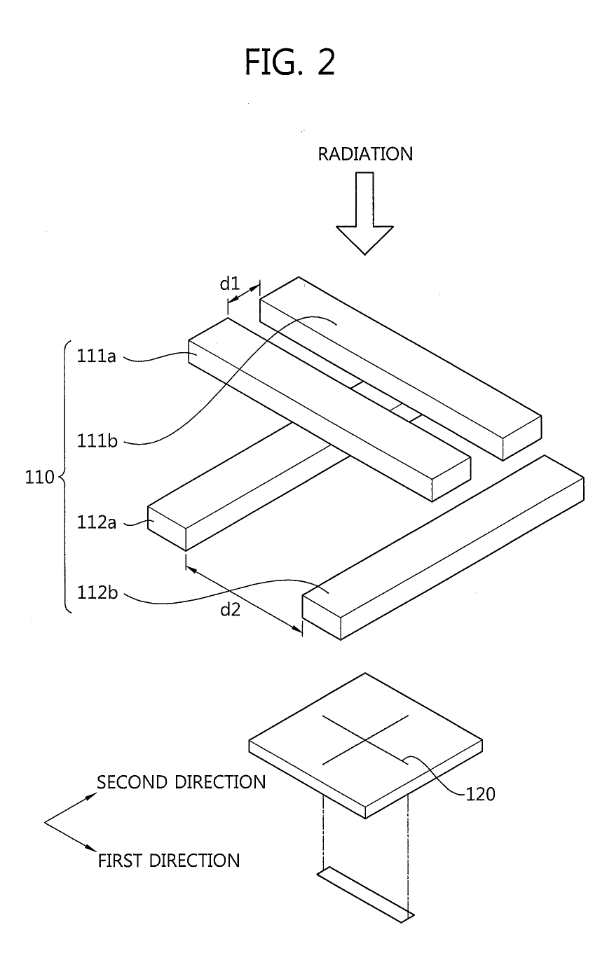

FIG. 2 is a perspective view illustrating a method for controlling size of radiation irradiation of a system for radiation therapy according to an exemplary embodiment of the present invention.

The radiation generated from a radiation source (not shown) passes through a radiation shaping unit 110, so that field size and direction of the radiation is controlled. The radiation having passed through the radiation shaping unit 110 then passes through a crosshair indicating a radiation field center 120 to be irradiated onto the patient. A light source (not shown) for optically alignment may be mounted in the gantry 100.

The radiation shaping unit 110 may be called as a collimator. The radiation shaping unit 110 includes a pair of first shaping parts 111a and 111b extended in a first direction and a pair of second shaping parts 112a and 112b extended in a second direction. Intervals d1 and d2 and directions of the first shaping parts 111a and 111b and the second shaping parts 112a and 112b may be controllable, so that the field size and direction of the radiation. The radiation shaping unit 110 may irradiate the radiation in a narrow rectangular shape, and the rectangular radiation field may be rotated.

The light for the alignment, which is generated from the light source (not shown), passes through the crosshair indicating a radiation field center 120 to be irradiated toward an exterior.

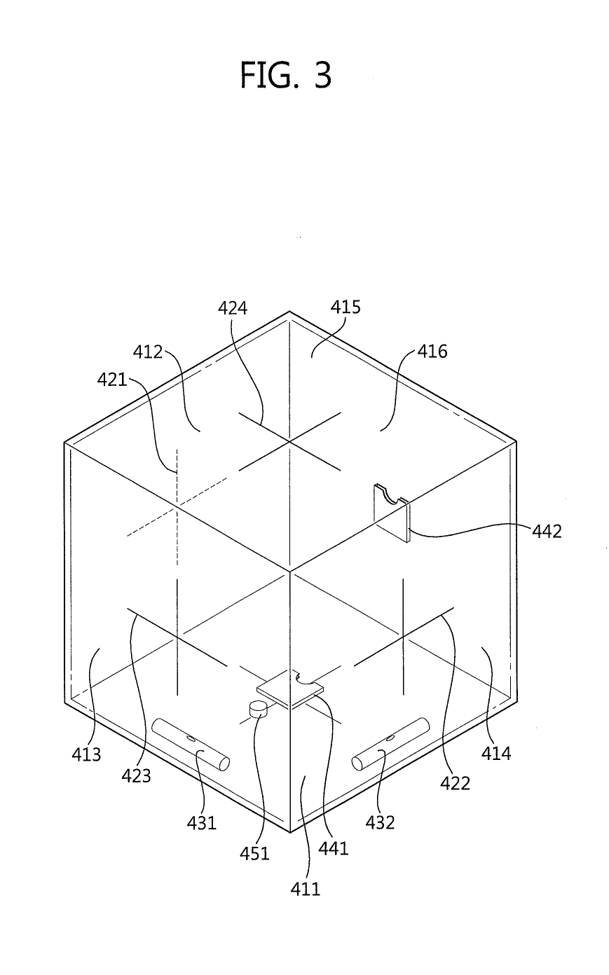

FIG. 3 is a perspective view illustrating an apparatus 400 for alignment according to an exemplary embodiment of the present invention.

The apparatus 400 for alignment has a rectangular parallelepiped shape having six faces 411 to 416.

The six faces 411 to 416 includes a lower face 411, side faces 412 to 415 and an upper face 416.

Two levels 431 and 432 may be disposed on the lower face 411, and may be arranged in substantially perpendicular to each other.

Alignment marks 421 and 422 may be disposed on both faces 412 and 414 facing each other. In the present exemplary embodiment, the both faces 412 and 414 include transparent plates. The alignment marks 421 and 422 include translucent that blocks light, and are printed or engraved at the transparent plates. The alignment marks 421 and 422 have different lines as a solid line and a broken line, so that images of the alignment marks 421 and 422 generated by the irradiation of the light may be distinguishable.

An alignment mark 423 is disposed on another face 413, and an alignment mark 424 is disposed on the upper face 416.

In the present exemplary embodiment, the alignment marks 421 to 424 may be a crosshair shape. In another exemplary embodiment, the alignment marks 421 to 424 may have various shapes to recognize the alignment status.

In the present exemplary embodiment, the translucent alignment marks 421 and 422 are disposed on the transparent plates, thereby generating the image of the light irradiation. In another exemplary embodiment, transparent alignment marks may be disposed on translucent plates.

An alignment light source 451 is disposed in an internal space of the rectangular parallelepiped. The alignment light source 451 may include a light emitting diode (LED) to irradiate light toward the alignment marks 423 and 424.

In the present exemplary embodiment, the radiation imaging part that recognize an isocenter of the radiation that is irradiated from the gantry 100 includes film holding parts 441 and 442, in which radiation-sensitive films are mounted. The film holding parts 441 and 442 include the plural radiation imaging planes. The film holding parts 441 and 442 include a first film holding part 441 and a second film holding part 442. The film is mounted in the first film holding part 441 to face a rotation plane of the radiation shaping unit 10. The film is mounted in the second film holding part 442 to face a rotation plane of the gantry 100. In the present exemplary embodiment, the film is mounted in the first film holding part 441 in the horizontal direction, and the film is mounted in the second film holding part 442 in the vertical direction. The first and second holding parts 441 and 442 are spaced apart from each other in the horizontal direction.

In another exemplary embodiment, the film holding parts 441 and 442 control the direction of the film by rotation.

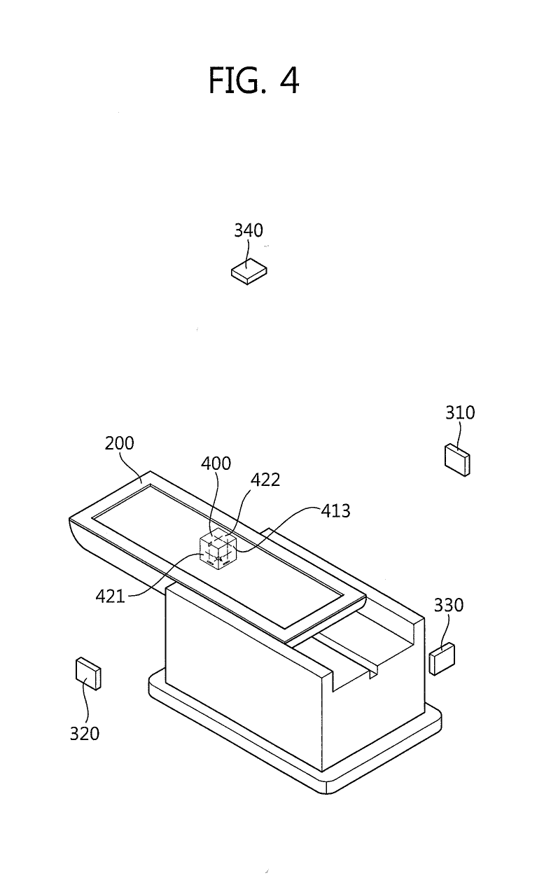

FIG. 4 is a perspective view illustrating the apparatus 400 for laser alignment disposed in the couch 200 according to an exemplary embodiment of the present invention.

The apparatus 400 for laser alignment is disposed at a position on which the patient receiving the radiation lies.

The alignment marks 421 and 422 facing each other correspond to the right and left of the patient, respectively. The alignment mark 421 faces the second laser unit 320, and the alignment mark 422 faces the first laser unit 310.

The alignment mark 423 corresponds to the feet of the patient, and faces the third laser unit 330 disposed at a direction of the feet of the patient.

The alignment 424 corresponds to an upper portion of the patient, and faces the fourth laser unit 340 disposed at the ceiling.

The above-mentioned apparatus 400 for alignment is disposed on the couch 200 at which the patient is disposed, thereby aligning the laser.

In the present invention, the laser alignment includes an optical alignment and a radiation alignment.

The optical alignment uses the crosshair indicating a radiation field center 120 and the alignment marks 421 to 424 of the apparatus 400 for laser alignment. The optical alignment aligns the laser device 310 to 340 using the optical image formed by the light irradiation of the light source onto the alignment marks and the alignment marks, thereby aligning the laser device 310 to 340. In the present exemplary embodiment, the optical image is generated by the shadow formed by the alignment marks.

In the radiation alignment, a radiation center is estimated from the radiation image irradiated onto the radiation film, and the laser device 310, 320, 330 and 340 are aligned with respect to the radiation center.

Hereinafter, the optical alignment is performed, and then the radiation alignment is performed. However, the scope of the present invention is not limited by the following method. The present invention also includes a method of performing a portion of the radiation alignment after performing the optical alignment, a method of only performing the optical alignment or the radiation alignment, etc.

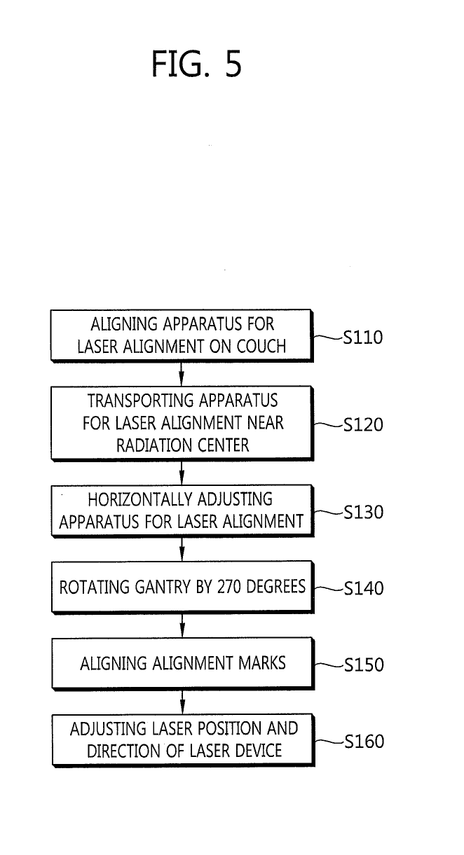

The optical alignment will be explained with reference to FIGS. 5 to 7.

FIG. 5 is a flow chart illustrating aligning a laser device 310 according to an optical method of an exemplary embodiment of the present invention.

Firstly, the apparatus 400 for laser alignment is disposed on the couch 200 as shown in FIG. 4 (step S110).

Then, the position of the center of the apparatus 400 of the laser alignment is controlled so that the center of the apparatus 400 of the laser alignment is disposed around the radiation center (step S120). Thus, disturbance of horizontal level of the apparatus 400 for the laser alignment, which is caused by excessive transportation of the couch 200 is prevented during the alignment.

Then, the apparatus 400 for laser alignment is horizontally leveled by the level (step S130). The horizontal level of the apparatus 400 for the laser alignment may be adjusted using a height controlling member (a level adjusting part, not shown) disposed in the apparatus 400 for laser alignment. Also, the horizontal level of the apparatus 400 for the laser alignment may be adjusted using the couch 200.

After the apparatus 400 for laser alignment is leveled, the gantry 100 is rotated toward 270 degree direction (step S140). The rotation of the gantry 100 may be performed before the leveling of the apparatus 400 for laser alignment. When the gantry 100 is rotated toward the 270 degree direction, the light source of the gantry 100 the crosshair indicating a radiation field center 120, the alignment marks 421 and 422 of the apparatus 400 for the laser alignment and the first laser unit 310 are substantially linearly aligned.

Then, the light source irradiates light in the gantry 100 to generate the optical image of the crosshair indicating a radiation field center 120 and the alignment marks 421 and 422, so that the optical image of the alignment marks are substantially coincident (step S150). The coincidence of the optical image is performed rotation or translation of at least one of the gantry 100, the couch 200 and the radiation shaping unit 310.

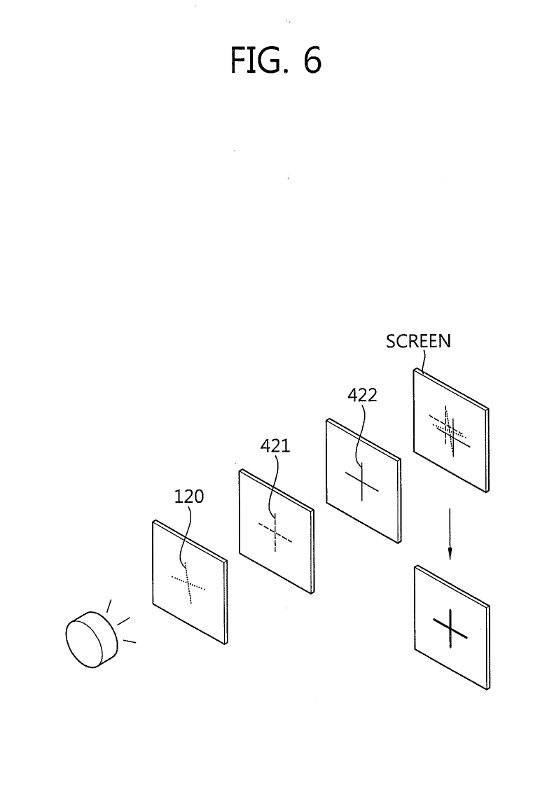

The above-mentioned processes will be explained with respect to FIG. 6. FIG. 6 is a perspective view illustrating coincidence of alignment mark of optical alignment according to an exemplary embodiment of the present invention.

As shown in FIG. 6, the light generated from the light source of the gantry passes through the crosshair indicating a radiation field center 120 and the alignment marks 421 and 422 of the apparatus 400 for the laser alignment, in sequence, to generate the optical image, i. e. a shadow, on a screen. In order to coincide the three shadows, at least one of the gantry 100, the couch 200 and the radiation shaping unit 310 is rotated or translated. The screen may include light and translucent paper, thin plastic plate, etc.

In the present exemplary embodiment, the alignment marks 421 and 422 of the crosshair indicating a radiation field center 120 and the apparatus 400 for laser alignment generate shadows having different thicknesses and shapes. Thus, the alignment marks 421 and 422 of the crosshair indicating a radiation field center 120 and the apparatus 400 for laser alignment may be easily distinguished and controlled.

Then, the position of the direction of the first laser unit 310 are controlled with respect to the coincided optical image (step S160).

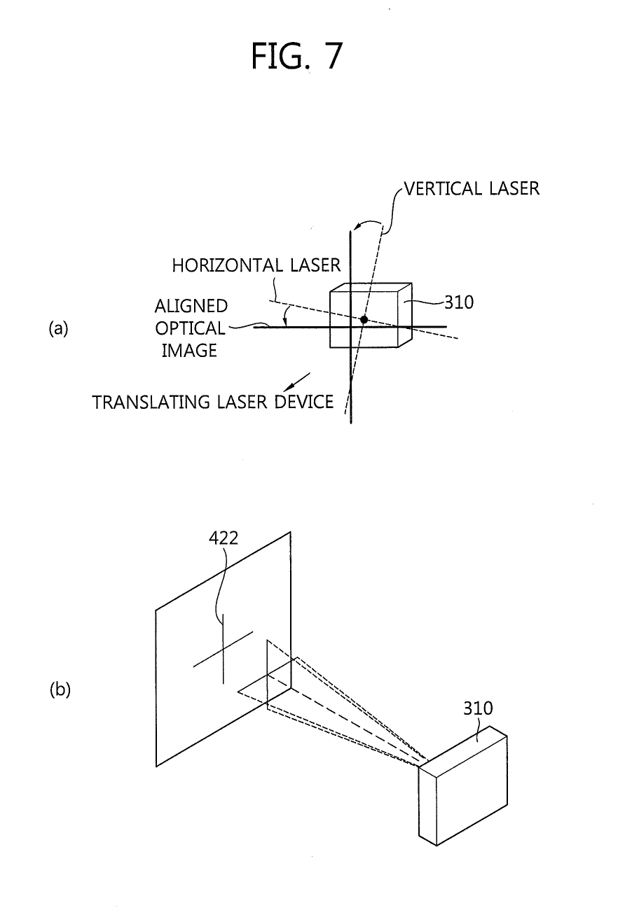

The above-mentioned process will be explained with respect to FIG. 7. FIG. 7 is a perspective view illustrating control of position and direction of a laser device according to an optical alignment of an exemplary embodiment of the present invention.

When the coincided optical image is displayed on the first laser unit 310, the position of the first laser unit 310 is transported as shown in (a) so that the vertical and horizontal lasers coincide with the optical image. As shown in (b), the direction of the vertical and horizontal lasers are controlled to be coincided with the first alignment mark 422 of the apparatus 400 for alignment, into which the laser is irradiated.

The optical alignment of the first laser unit 310 explained with reference to FIGS. 5 to 7 may be applied to the optical alignment of the second laser unit 320 with respect to the gantry 270 of 90 degree position.

The optical alignment of the third laser unit 330 and the fourth laser unit 340 may be performed by the above-mentioned optical alignment method. The difference between the optical alignment of the first and second laser units 310 and 320 and the optical alignment of the third laser unit 330 and the fourth laser unit 340 is that the alignment light source 451 in the apparatus for alignment is used instead of the light source in the gantry and one alignment mark is used.

The third laser unit 330 or the fourth laser unit 340 is disposed above a shadow of the third alignment mark 423 or the fourth alignment mark 424, which is formed by the alignment light source 451, and the direction of the sagittal laser is controlled to be coincided with the third alignment mark 423 or the fourth alignment mark 424.

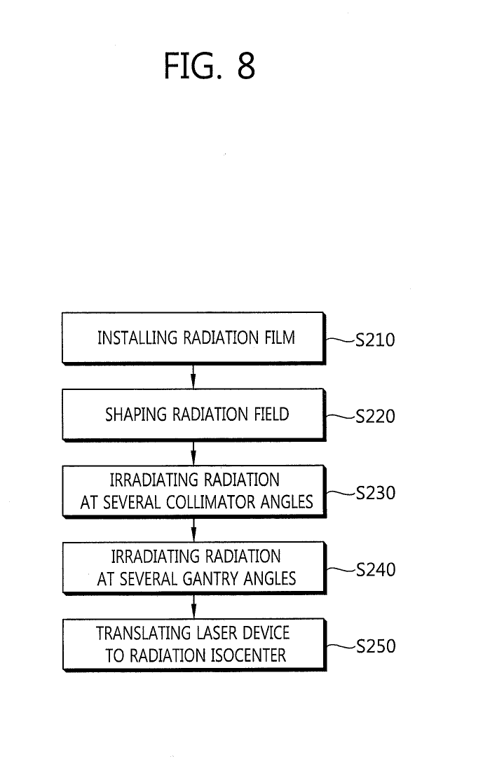

The radiation alignment will be explained with reference to FIGS. 8 to 11.

FIG. 8 is a flow chart illustrating radiation alignment according to an exemplary embodiment of the present invention.

Firstly, radiation films 441a and 442a are received in the film holding parts 441 and 442 (step S210). The radiation films 441a and 442a include radiation sensitive material that changes color by irradiation of radiation, so that a portion exposed by the radiation may be displayed.

Then, the radiation shaping unit 110 is controlled to control field size and direction of the radiation (step S220). Preferably, the radiation used for the radiation alignment may have a rectangular shape. Also, the radiation shaping unit 110 is controlled so that the radiation is not irradiated onto the second radiation film 442a but only irradiated onto the first radiation film 441a horizontally arranged.

Then, the radiation shaping unit 110 rotates and irradiates radiation while the gantry 100 is fixed at a 0 degree position (step S230). The radiation shaping unit 110 may be intermittently or continuously rotated.

Then, the radiation shaping unit 110 is controlled so that the radiation is not irradiated onto the first radiation film 441 a but irradiated onto the second radiation film 442a. The gantry 100 rotates to radiate the radiation while the radiation controlling unit 110 is fixed (step S240). The gantry 100 may be intermittently or continuously rotated.

The two steps (S230 and S240) of the above-mentioned irradiation of the radiation may be changed.

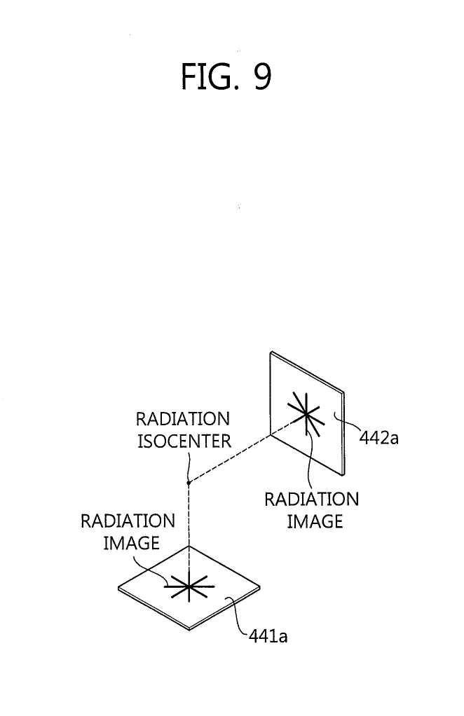

The radiation image formed by the irradiation of the radiation will be explained with reference to FIG. 9. FIG. 9 is a radiation image irradiated on a radiation film according to an exemplary embodiment of the present invention.

When the radiation shaping unit 110 intermittently rotates by a constant angle to irradiate the radiation four times, the radiation image such as shown in the first radiation film 441a may be formed. Also, when the gantry 100 intermittently rotates by a constant angle to irradiate the radiation four times, the radiation image such as shown in the second radiation film 442a may be formed. The radiation shaping unit 110 is controlled at each step so that the position of the radiations are spaced apart from each other. Thus, the radiation of a following step does not affect the radiation image of a previous step.

Centers of the radiation images of two radiation films 441a and 442a are extended in a perpendicular direction to get an intersecting point as an isocenter of the radiation.

Then, the laser device 310 to 340 is translated with respect to the isocenter of the radiation (step S250).

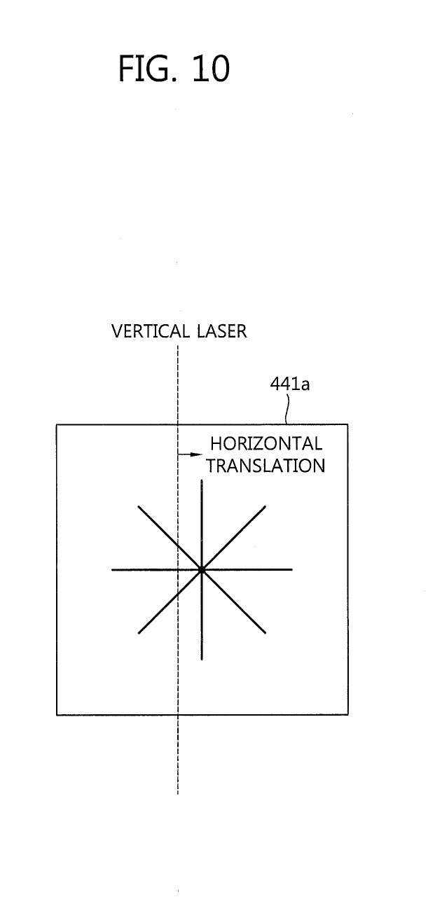

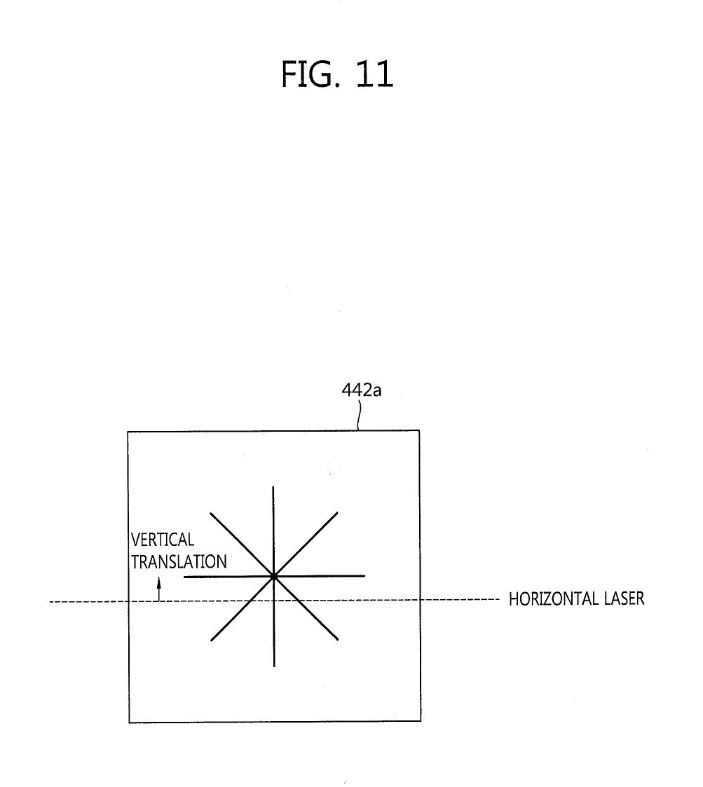

The above-mentioned step will be explained with reference to FIGS. 10 and 11. FIG. 10 is a side view illustrating a vertical laser alignment of a radiation alignment according to an exemplary embodiment of the present invention. FIG. 11 is a plan view illustrating a horizontal laser alignment of a radiation alignment according to an exemplary embodiment of the present invention.

The center of the radiation image in the first radiation film 441a becomes a reference for the alignment of the vertical laser alignment. The laser device 310 to 340 is horizontally translated as shown in FIG. 10, so that the vertical laser of each laser unit 310 to 340 cross the center of the radiation image on the first radiation film 441a.

The center of the radiation image in the second radiation film 442a becomes a reference for the alignment of the horizontal laser alignment. The laser device 310 to 340 is vertically translated as shown in FIG. 11, so that the horizontal laser of each laser unit 310 to 340 cross the center of the radiation image on the second radiation film 442a.

By the above-mentioned steps, the lasers of each layer unit 310 to 340 cross the isocenter of the radiation while the lasers of each laser unit 310 to 340 are completely overlapped. Thus, the treatment site for treatment may be exactly pointed with respect to the reference point of the radiation. Therefore, exact radiation treatment may be performed.

In the present invention, the optical alignment uses the plural alignment marks 421 and 422. When the optical alignment uses two alignment marks 421 and 422, laser alignment error is greatly decreased compared with a conventional method although error in the level measurement or the position error of the light source is formed.

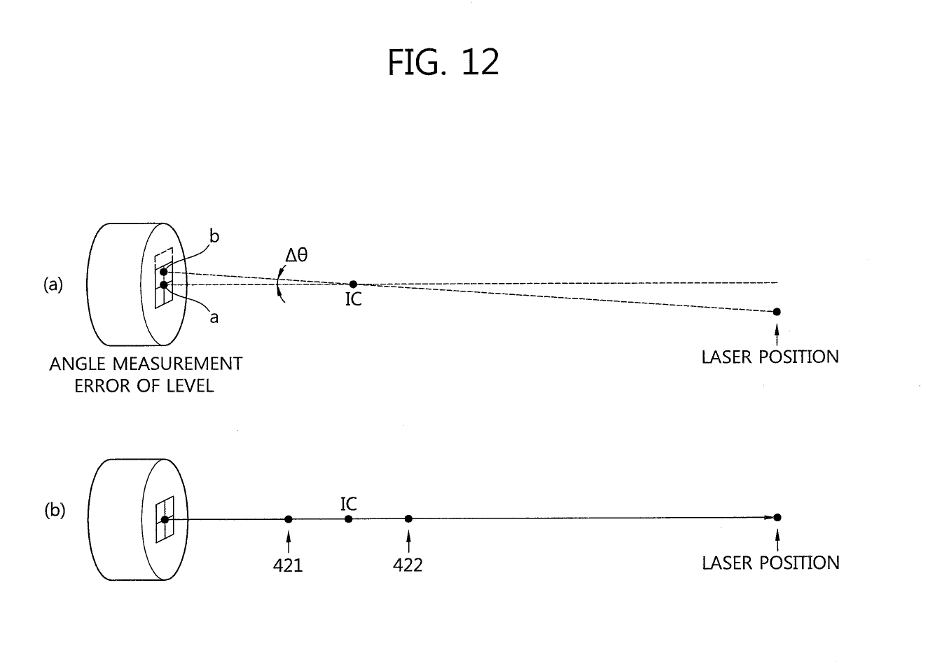

FIG. 12 is a perspective view illustrating difference of a horizontal measurement error between a related art and the present invention.

When the angle measurement error (Δθ, ‘a’ represents angle measurement without error and ‘b’ represents angle measurement having error) of the level is formed in the prior art (a), the laser is dislocated from the radiation central line IC to greatly increase the error of the laser position. However, in the present invention (b), the alignment marks 421 and 422 disposed on both sides of the radiation center IC to remove the generation of the error of the level measurement.

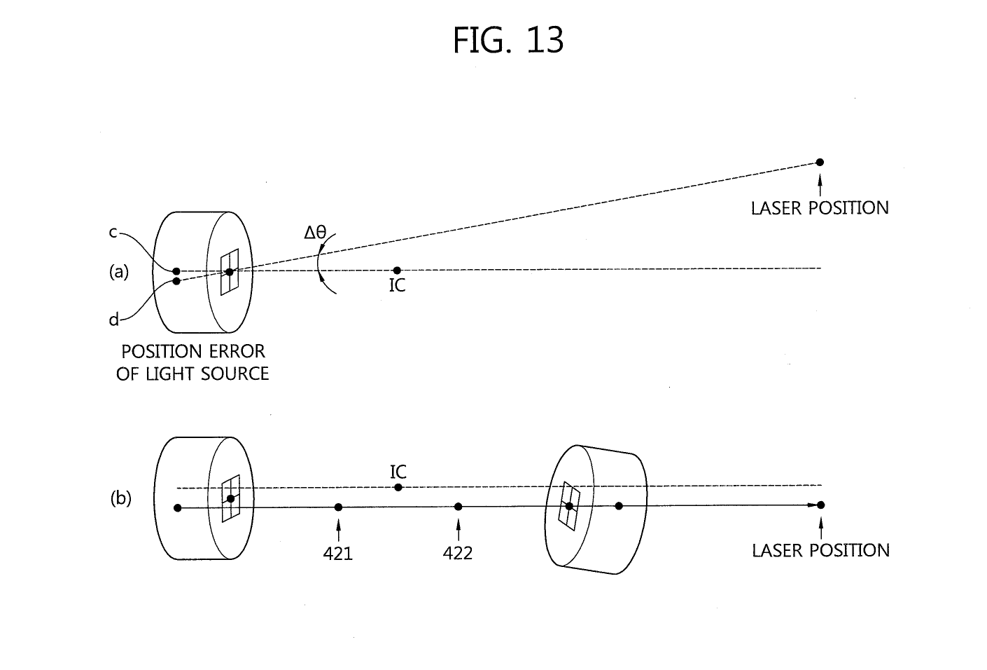

FIG. 13 is a perspective view illustrating difference of an optical error between a related art and the present invention.

In the prior art (a), when the position error of the light source Δθ is occurred, the position error (‘c’ represents a point without the position error, and ‘b’ represents a point with the position error) fully affects the laser alignment. In contrast, in the present invention (b), the laser is parallelly aligned with the central line IC of the radiation by the plural alignment marks 421 and 422. Then, the radiation alignment is performed to accurately align the laser.

The conventional method of the laser alignment increases accuracy of the laser alignment by repetitively aligning processes. However, the method of the laser alignment of the present invention obtains substantially the same or more accurate laser alignment by only one aligning process. Thus, the time for the laser alignment is greatly decreased.

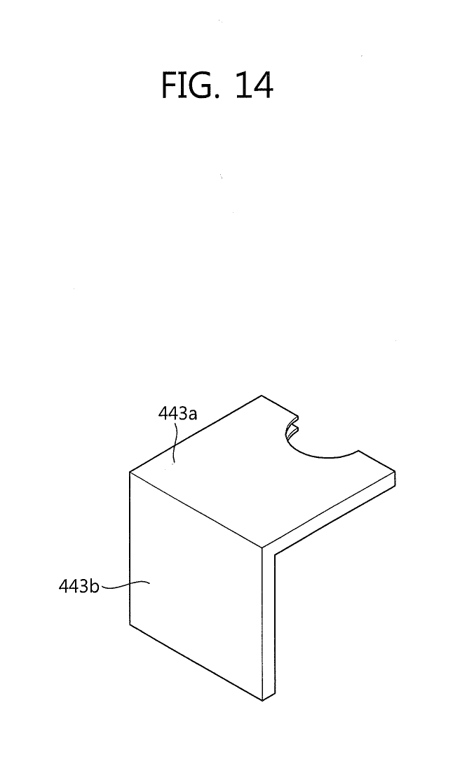

FIG. 14 is a perspective view illustrating a radiation imaging part according to an exemplary embodiment of the present invention.

In the present exemplary embodiment, the radiation imaging part includes single film holding part 443. The film holding part 443 may receive a radiation sensitive film therein, and has a first imaging plane 443a and a second imaging plane 443b that are disposed in different planes.

In another exemplary embodiment, the radiation imaging part may include or receive at least one of radiation image detectors, such as a gel dosimeter, a fluorescent substance, a scintillator and an imaging device using a photovoltaic effect.

Also, the radiation imaging part may include the plural independent radiation imaging planes spaced apart from each other, and may be disposed on a surface of a single object such as a cylinder, a sphere, an ellipsoid or a hexahedron. In addition, the radiation imaging plane may have a curved shape, and the radiation imaging part may control angle by rotation.

The foregoing is illustrative of the present teachings and is not to be construed as limiting thereof. Although a few exemplary embodiments have been described, those skilled in the art will readily appreciate from the foregoing that many modifications are possible in the exemplary embodiments without materially departing from the novel teachings and advantages of the present disclosure of invention. Accordingly, all such modifications are intended to be included within the scope of the present teachings. In the claims, means-plus-function clauses are intended to cover the structures described herein as performing the recited function and not only structural equivalents but also functionally equivalent structures.

What is claimed is:

1. An apparatus for alignment used in a radiation therapy system, the radiation therapy system including a rotatable gantry having a radiation source generating radiation, a radiation shaping unit and a crosshair indicating a radiation field center, a couch on which a patient is disposed and a laser device disposed adjacent to the gantry to irradiate a laser for aiming, a light source irradiating light being mounted in the gantry, the couch being disposed adjacent to the gantry, position and direction of the couch being controllable, the apparatus for alignment comprising:
the plural alignment marks spaced apart from each other to be projected, thereby checking alignment status by the irradiation of the light in at least one direction;
a level indicating horizontal level of the alignment marks; and
a level adjusting part adjusting horizontal level of the alignment marks.

2. The apparatus for alignment of claim 1, further comprising a radiation imaging part including the plural radiation imaging planes to check an isocenter of the radiation irradiated by the gantry.

3. The apparatus for alignment of claim 2, wherein the radiation imaging plane comprises:
a first imaging plane directly facing a rotation plane of the gantry or facing the rotation plane of the gantry by rotation; and
a second imaging plane directly facing a rotation plane of the radiation shaping unit or facing the rotation plane of the radiation shaping unit by rotation.

4. The apparatus for alignment of claim 2, wherein the radiation imaging part comprises or receive at least one of radiation image detectors, such as a gel dosimeter, a fluorescent substance, a scintillator and an imaging device using a photovoltaic effect, and the radiation is irradiated onto the radiation imaging part through the radiation shaping unit.

5. The apparatus for alignment of claim 1, wherein the laser device comprises first and second laser units disposed on left and right of the gantry,
the apparatus for alignment further comprises first and second plates facing each other,
the alignment mark includes a first alignment mark formed on the first plate and a second alignment mark formed on the second plate, and
the first and second alignment marks are used for aligning the first and second laser units with the light source mounted in the gantry.

6. The apparatus for alignment of claim 5, wherein the alignment mark has a crosshair shape, and
the first and second alignment marks have different linear shapes different from each other.

7. The apparatus for alignment of claim 5, further comprising an alignment light source irradiating light toward the alignment mark.

8. The apparatus for alignment of claim 7, wherein the laser device further comprises a third laser unit disposed on a side facing the gantry,
the apparatus for alignment further comprises a third plate nonparallel from the first and second plates and facing the third laser,
the alignment mark further includes a third alignment mark formed on the third plate, and
the third alignment mark is used for aligning the third laser unit with the alignment light source.

9. The apparatus for alignment of claim 8, wherein the laser device further comprises a fourth laser unit disposed above the gantry,
the apparatus for alignment further comprises a fourth plate nonparallel from the first to third plates and facing the fourth laser,
the alignment mark further includes a fourth alignment mark formed on the fourth plate, and
the fourth alignment mark is used for aligning the fourth laser unit with the alignment light source.

10. A method for laser alignment used in a radiation therapy system, the radiation therapy system including a rotatable gantry having a radiation source generating radiation, a radiation shaping unit and a crosshair indicating a radiation field center, a couch on which a patient is disposed and a laser device disposed adjacent to the gantry to irradiate a laser for aiming, a light source irradiating light being mounted in the gantry, the couch being disposed adjacent to the gantry, position and direction of the couch being controllable, the method for laser alignment comprising:
preparing an apparatus for alignment including first and second alignment marks spaced apart from each other to be projected to simultaneously checking alignment status by the irradiation of the light in at least one direction, a level and a level adjusting part;
disposing the apparatus for alignment on the couch;
transporting the apparatus for alignment toward a radiation center to level the apparatus for alignment;
rotating or translating the gantry, the radiation shaping unit and the couch while irradiating the light of the light source in the gantry to coincide the first and second alignment marks with an optical image of the crosshair indicating a radiation field center; and
locating the laser device on an optical image formed by coinciding the first and second alignment marks, and controlling direction of the laser so that the first and second alignment marks are coincided with the laser.

11. The method of claim 10, wherein the alignment mark has a crosshair shape, and
the first and second alignment marks have different linear shapes different from each other.

12. The method of claim 10, further comprising checking an isocenter of the radiation irradiated from the gantry to translate the laser or the laser device.

13. The method of claim 12, wherein the apparatus for alignment further comprises a radiation imaging part having the plural image surfaces, and
the isocenter of the radiation irradiated from the gantry is checked using radiation image obtained by the radiation imaging part.

14. The method of claim 13, wherein the radiation imaging plane comprises:
a first imaging plane directly facing a rotation plane of the gantry or facing the rotation plane of the gantry by rotation; and
a second imaging plane directly facing a rotation plane of the radiation shaping unit or facing the rotation plane of the radiation shaping unit by rotation.

15. The method of claim 14, wherein the isocenter of the radiation is checked by:
rotating the gantry to irradiate the radiation onto the first imaging plane while the radiation shaping unit is fixed; and
rotating the radiation shaping unit onto the second imaging plane while the gantry is fixed.

16. A method for laser alignment used in a radiation therapy system, the radiation therapy system including a rotatable gantry having a radiation source generating radiation, a radiation shaping unit and a crosshair indicating a radiation field center, a couch on which a patient is disposed and a laser device disposed adjacent to the gantry to irradiate a laser for aiming, a light source irradiating light being mounted in the gantry, the couch being disposed adjacent to the gantry, position and direction of the couch being controllable, the method for laser alignment comprising:
preparing an apparatus for alignment including first and second alignment marks spaced apart from each other to be projected to simultaneously checking alignment status by the irradiation of the light in at least one direction, a level and a level adjusting part;
optically aligning the laser device using optical image obtained by lights having substantially simultaneously passed through the crosshair indicating a radiation field center, the first alignment mark and the second alignment mark; and
determining center of the radiation irradiated from the gantry to align the laser device.

17. The method of claim 16, wherein the alignment mark has a crosshair shape, and
the first and second alignment marks have different linear shapes different from each other.

18. The method of claim 16, wherein the apparatus for alignment further comprises a radiation imaging part having the plural image surfaces, and
the isocenter of the radiation irradiated from the gantry is checked using radiation image obtained by the radiation imaging part.

19. The method of claim 18, wherein the radiation imaging plane comprises:
a first imaging plane directly facing a rotation plane of the gantry or facing the rotation plane of the gantry by rotation; and
a second imaging plane directly facing a rotation plane of the radiation shaping unit or facing the rotation plane of the radiation shaping unit by rotation.

20. The method of claim 19, wherein the isocenter of the radiation is checked by:
rotating the gantry to irradiate the radiation onto the first imaging plane while the radiation shaping unit is fixed; and
rotating the radiation shaping unit onto the second imaging plane while the gantry is fixed.
